(19) United States
(12) Patent Application Publication
(10) Pub. No.: US 2015/0025307 A1
(43) Pub. Date: Jan. 22, 2015

(54) DEVICE USED IN THE CONTEXT OF A GYNECOLOGICAL TRANSFER AND THE MANUFACTURE OF SUCH A DEVICE

(71) Applicant: Prodimed, Neuilly-en-Thelle (FR)

(72) Inventors: Patrick Bouveret, Neuilly en Thelle (FR); Patrick Choay, Paris (FR)

(21) Appl. No.: 14/375,605

(22) PCT Filed: Jan. 30, 2013
(86) PCT No.: PCT/FR2013/050185

§ 371 Date: Jul. 30, 2014

(30) Foreign Application Priority Data
Feb. 1, 2012 (FR) 1250941

(51) Int. Cl. A61B 17/435; A61B 17/43

(52) U.S. Cl. CPC A61B 17/435; A61B 17/43
USPC 600/34; 600/35

(57) ABSTRACT

This invention concerns a device for gynaecological transfer or insemination with shape memory comprising a catheter comprising a polymer tube open at its ends, characterised in that this tube is comprised of a medium-density polymer associated with mineral filler.

Such a device presents an adaptable diameter and a structure not enlarged by the presence of a metallic mesh without imposing fastidious handling such as can be the case of a flexible introducer catheter associated with a metallic rod for positioning.

This invention concerns a medical device used in gynaecology and more particularly a device for the transfer of embryos or artificial insemination equipped with a catheter with a material that is both malleable (bendable) and capable of keeping the shape given after bending.

Such materials thus are able to change shape following pressure imposed by the user, and to retain the imposed shape. Bending is said to be inelastic or plastic.

For convenience, these materials are said to have “shape memory”.

Current transfer devices, whether designed for sampling or injection/insemination or embryo transfer, generally comprise a catheter in the form of a hollow cylindrical tube open at both ends and small in diameter. For the transfer, this tube is linked or associated by one of its ends, to example via a connector, to a device used to create a depression or exert moderate pressure to perform a sampling or injection respectively.

In one design variation, some devices comprise on the one hand an introducer catheter and, on the other hand, a transfer catheter. This purpose of this design mode is the insertion of the introducer catheter in the uterine cavity. Typically, within the framework of an embryo transfer, this catheter is inserted in the uterine cavity. Once the introducer catheter is correctly positioned, the transfer catheter that contains the embryo to be transferred, which has a smaller external diameter than the introducer catheter, is inserted in this introducer catheter and directly enters the area desired for the transfer.

According to this design method, the transfer catheter is associated with the moderate pressure or depression device. This design method is generally used to bring the transfer catheter directly into position without risk of contaminating the body parts crossed and to facilitate introduction of the catheter in the event of a cervix that may be difficult to catheterise.

During embryo transfer or insemination, for the catheter to directly enter the uterine cavity in the ideal location, medical professionals must pass the catheter through the cervix. In many cases, however, the cervix is not in line with the uterus and a special catheter is required to reach the uterine cavity.

Therefore, there exist bendable catheters in flexible polymer, for example, such as those described in the patent U.S. Pat. No. 6,027,443A, which are extremely flexible. This catheter prevents trauma to internal uterine wails while presenting the flexibility and texture allowing insertion along complex paths, as the catheter bends when it meets a wall without damaging it.

However, this flexibility requires very careful handling of the device requiring numerous manoeuvres to position the catheter correctly, as the direction of the catheter when it folds cannot be easily directed. Rapidity of catheter insertion and the absence of traumatism to the uterine cavity are key factors in successful transfer.

To prevent this, there exist catheters that can be pre-shaped that retain their shape before introduction in the uterus and that can be directed directly according to paths determined by the medical professional. Typically, the medical professional examines the patient to determine the path to take for the catheter and then gives this catheter the required shape so that it follows this path precisely without hitting the internal walls of the patient.

So that such a catheter can be pre-shaped, a rod forming a metallic mesh or a metallic alloy, as described in the patent WO 2006/011127A2, is generally incorporated in the catheter tube. This metallic mesh gives the tube the rigidity and shape memory capacity allowing the medical professional to modulate the tube shape as needed.

This type of known catheter however requires special handling to ensure the shape is retained and often presents a risk of folding when the rod is withdrawn, making the catheter unusable. In addition, the integration of such a metallic rod in the walls of the catheter significantly increases the diameter of the catheter thus reducing patient comfort and could provoke uterine contractions.

Within the framework of an introducer catheter associated with a transfer catheter, it is possible to provide a malleable metallic rod with related shape memory. During use, the health-care professional inserts the metallic rod in the introducer catheter and gives the rod and introducer catheter pair the desired shape. The catheter retains this shape imposed by the metallic rod that it contains during positioning of the pair in the uterus. Once the introducer catheter is correctly positioned, the metallic rod is withdrawn to introduce the transfer catheter in its place and perform the embryo transfer for example.

This method is the most often used at present but it requires, on the one hand, the manufacture of an independent malleable metallic rod and on the other, additional handling to insert the metallic rod and remove It to be able to use the transfer catheter which can generate asepsis faults.

The problem uncovered by these techniques is that of creating a catheter that is both sufficiently flexible to be bent at will without breaking or folding by the medical professional, and able to retain the shape given in this way. Prior-art catheters do not manage to satisfy these contradictory characteristics in an optimal way.

The purpose of the invention is therefore to provide a malleable (bendable) catheter with shape memory, non-elastic bending properties and bendable at will, that retains the shape given by the medical professional in the uterus. The catheter must moreover remain easy to use without requiring additional handling and not be fastidious to install or position.

The purpose of the invention is therefore to provide a malleable gynaecological transfer device with shape memory comprising a catheter comprising a polymer tube open at both ends, characterised in that this tube is composed of a medium-density polymer associated with mineral filler.

A medium-density polymer as used in the composition of the catheter tube according to this invention presents characteristics used to obtain a flexible catheter that is bendable in a simple and desired way that is not too flexible as are prior-art devices in polymer.

This medium-density polymer can be directly selected from medium-density polymers or obtained by a mixture of high-density polymer and low-density polymer; such a mixture is created beforehand to obtain a homogeneous material for its use in the invention.

Preferably, the mixture comprising the catheter tube should include between 60% and 90% medium-density polymer and respectively between 40% and 10% mineral filler. Ideally, the tube comprises approximately 80% polymer and 20% mineral filler. Percentages are expressed in weight.

To reduce and control the elasticity of the medium-density polymer, the latter is mixed with mineral filler, whose molecules insert themselves between the molecules of the medium-density polymer and reduce its elasticity. This mineral filler mechanically reduces the elasticity and increases the malleable character (bending) of the plastic; the catheter thus comprises an amorphous tube bending at will and retaining the use of the given shape.

Preferably, the medium-density polymer is a polyolefine. This polyolefine is easily transformable and can be used to manufacture a particularly malleable tube. Advantageously, this polyolefine is a polyethylene; the tube obtained from this polyethylene is thus more flexible and easier to transform.

In special design modes, the mineral filler can be titanium dioxide lending a pearly look and good sliding characteristics to the tube. In one variant, the mineral filler is a barium sulphate, which improves the malleability of the tube. If appropriate, this mineral filler can be bismuth salt; these mineral fillers can be used to obtain a tube with satisfactory gliding properties.

To obtain special visual effects or adapt the sliding and malleability characteristics of the tube, the polymer can be mixed with different mineral fillers to form the tube.

The invention also deals with a manufacturing procedure of a device according to the invention characterised in that it comprises a prior stage of mixing a medium-density polymer and a mineral filler, for example by fusion or “compounding” (manufacture of a compound/mix of plastic materials), followed by an extrusion phase of products obtained during the mixing or “compounding”.

This tube manufacturing procedure should be performed by extrusion but can also be achieved by moulding or any other means known to experts.

The step of mixing the medium-density polymer with the mineral filler by compounding is used to obtain granules of homogenous mixed materials from these components. Thus, during an extrusion step used to manufacture the tube itself, the granules introduced in the extruder obtained by the prior mixing phase are homogeneous in composition and are used to obtain a tube by extrusion with the same malleability, flexibility and shape retention characteristics irrespective of the location of this tube.

In a preferable production mode, the mix comprises 81.5% of average-density polyethylene, such as for example, Lupolen®3721C, 15% BaSo4, 3% TiO2 and 0.5% colouring, for example, sea blue colouring.

The percentages of average-density polyethylene can vary from 75% to 85%; the BaSO4 from 15 to 20%; 2 to 3% TiO2; and 0.2 to 0.5% colouring, It is possible to use another average density PE (or a mix of high-density polyethylene PEHD and low-density polyethylene PEBD) on condition that the end product is comprised of at least 20% mineral filler.

The percentages above are given in weight.

Advantageously, extrusion take place according to four hot zones at 215° C., 226° C., 230° C. and 224° C. respectively, with a screw rotating at 27.1 rotations/min.

The temperatures indicated can vary according to the machine used, the flow rate desired and the tube's diameters. The transformation temperature range of the material is generally between 180 and 240° C.

The present invention is now described using purely illustrative examples that in no way limit the range of the invention, based on the following figures:

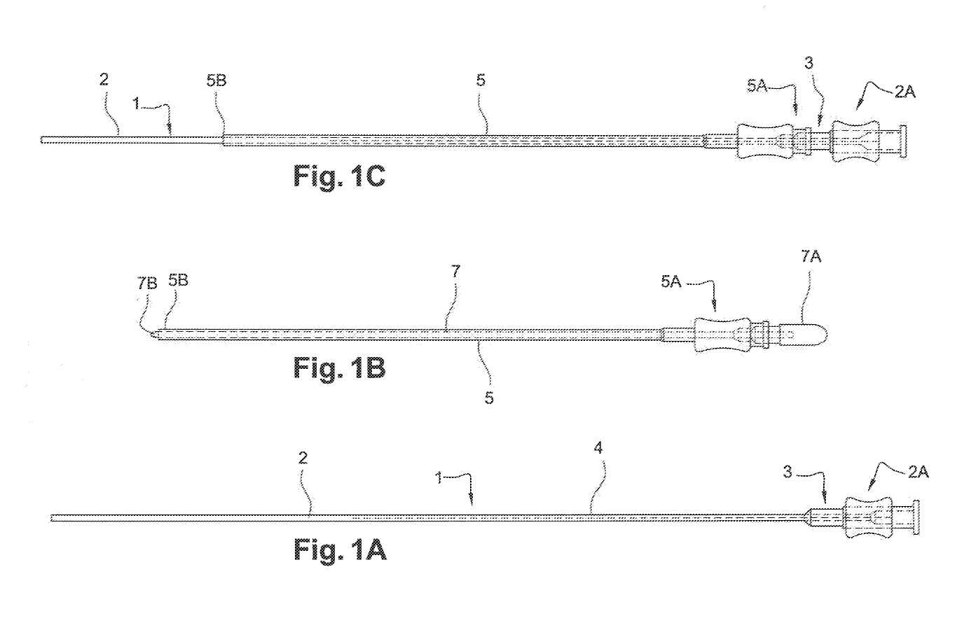

FIG. 1A is a schematic view from above showing a transfer catheter tube.

FIG. 1B is a schematic view from above showing an introducer catheter or tube. The catheter is equipped with a rod stopper which prevents mucus from entering the catheter.

FIG. 1C is a view of the introducer catheter of FIG. 1B (after removal of the rod stopper), in which the transfer catheter of FIG. 1A is introduced.

The transfer catheter 1 (FIG. 1A) is comprised of a cylindrical tube with a diameter in the order of 1.6 mm, and whose proximal end 2A comprises a gripping part 3 known itself. A metallic cannula 4 known itself is placed In tube 2.

The introducer catheter according to the FIG. 1B comprises in a known manner a tube 5 of approximately 2.7 mm in which is introduced a rod stopper 7 comprising a male element penetrating the interior of the tube 5 forming the introducer catheter.

The proximal end 7A of the rod 7 is supported by the proximal end 5A of the tube 5. and the distal end 7B of the rod 7 exceeds in the order of 2 mm in length the open distal end 5B of the tube 5. The rod stopper 7 is withdrawn from the tube 5 to be replaced by the transfer catheter 1 to perform the transfer.

The distal part 5B of the tube 5, designed to enter the patient's body, comprises mark lines regularly spaced in the order of 10 mm, for example, mark lines of 1 to 6. These mark lines are used by the medical professional to assess the length of the catheter entering the uterus of the patient.

In FIG. 1C, the transfer catheter 1 is represented inserted in the catheter or introducer tube 5 and exceeds a length of approximately 50 mm with respect to the open end 5B of the tube 5 forming the introducer catheter.

The gripping part 3 at the proximal end 2A of the transfer catheter 1 stops against the proximal end 5A (away from the patient) of the introducer catheter.

Within the framework, par example, of an embryo transfer or an insemination, the catheter must be inserted in the uterus of the patient such that the first end is accessible to the medical professional and therefore located outside of the uterus where the other end must pass through the cervix of the patient.

Depending on whether only a catheter is to be inserted in the uterine cavity and connected to the transfer device or if it involves the use of an introducer catheter that, once correctly positioned in the uterus, is intended to receive a transfer catheter, the catheter has a diameter respectively of between 0.7 mm and 1.60 mm or between 1.90 mm and 2.70 mm.

As the cervix of the uterus is rarely in line with the uterine cavity, the catheter tube (alone or introduction) must be sufficiently flexible to accommodate certain curves to reached the uterine cavity during its installation, that is, the tube must be able to follow the path from the outside of the uterus to the uterine cavity despite the change of direction imposed by passing through the cervix.

The catheter tube according to the invention is manufactured with medium-density polymer. The choice of medium-density polymer is made either directly in the polymers belonging to the medium-density polymer family, or by mixing a high-density polymer and a low-density polymer so as to obtain a polymer mix whose medium-density is that of a medium-density polymer, that is between 0.926 g/cm³ and 0.940 g/cm³.

This medium-density polymer is flexible enough to allow the tube to bend without risk of breaking or folding, and without being too rigid, which would require great effort to bend the tube and could be traumatic for the patient. Inversely, the polymer is not too flexible thus preventing random and undesirable folding of the tube which would require additional handling to position the catheter.

This medium-density polymer, however, keeps a given form when it is curved. Typically, its malleability properties are limited and in the absence of constraints, it tends to return to its original shape, generally rectilinear.

To retain the flexibility adequate to allow curving of the tube without breaking while removing/reducing the elasticity of the polymer tube which tends to return to its initial shape in the absence of constraints, the tube is shaped with a medium-density polymer associated with mineral filler. This mineral filler enters the composition of the tube from 10 to 40% with the medium-density polymer representing between 90 and 60% of the composition of the tube. These proportions, expressed in weight, are used advantageously to keep good tube flexibility along with sufficient rigidity to retain, throughout the embryo transfer or insemination, the shape given to the tube by the medical professional before insertion.

During manufacturing of the tube, the molecules of the mineral filler insert themselves between the molecules of the medium-density polymer and also reduce or even eliminate the natural elasticity of the medium-density polymer. Use, as in prior art, of either a high-density polymer or low-density polymer could make the tube breakable and not allow if to retain its shape after folding.

Advantageously, according to the invention, the medium-density polymer is a polyolefine, which is easier to process and work with for the manufacture of the tube than other polymers. Moreover, the polyolefines present features leading to a highly malleable tube for the medical professional who wishes to give the desired shape to the tube. This polyolefine is more exactly a polyethylene; the tube obtained is thus more flexible and easier to transform.

The mineral filler can be titanium dioxide, for its sliding qualities, barium sulphate or bismuth salt for its malleability.

Advantageously, the mineral filler is a mixture comprised of a number of mineral fillers with desirable properties, for example, a mixture of barium sulphate and titanium dioxide. In this way, in one design example, the tube according to the invention comprises 81.5% lupolen 3721C, average-density polymer of 0.9375 g/cm³, 3% titanium dioxide, 15% barium sulphate and 0.5% blue colouring.

This composition has the advantage of presenting non-elastic properties giving an “amorphous” material bending at will and retaining, in the uterus, the shape given it by the medical professional after examination of the patient while presenting, thanks to the mixture of mineral fillers, good sliding and malleability qualities.

The manufacturing process of a catheter tube according to the invention is described below within the framework of a tube manufactured by extrusion, but this tube could be manufactured any other way known to experts, for example by moulding.

To achieve a homogeneous tube presenting the desired features all along its length, the procedure according to the invention provides for a mixing of components prior to the extrusion. This mixing step is important, in particular if the manufacturing process of the tube cannot guarantee the homogeneous mixture of the components of the tube.

According to the invention, this is achieved by compounding or mixing by merging and preparing a semi-finished product in the form of granules constituting a homogeneous mixture of the components in the finished product.

The semi-finished product thus obtained comprising in the example given 81.5% Lupolen 3721C, 15% barium sulphate, 3% titanium dioxide TiO₂ and 0.5% colouring for example, sea blue, is presented in the form of granules directly useable by an extruder used to manufacture catheter tubes.

This extruder would comprise for example four hot zones at 215° C., 226° C., 230° C. and 224° C. respectively with a screw rotating at 27.1 rotations/min. The temperatures indicated can vary according to the machine used, the flow rate desired and the tube's diameters. The transformation temperature range of the material is generally between 180 and 240° C.

1. A device for inserting a malleable catheter within the framework of a gynaecological transfer or insemination bending at will while retaining, in the uterus, the shape given by the medical professional comprising a catheter comprising a polymer tube open at each end, wherein this tube comprises a medium-density polymer associated with mineral filler.

2. The device according to claim 1, wherein the polymer is a polyolefine.

3. The device according to claim 1, wherein the polymer is a polyethylene.

4. The device according to claim 1, wherein the mineral filler is of titanium dioxide.

5. The device according to claim 1, wherein the mineral filler comprises barium sulphate.

6. The device according to claim 1, wherein the mineral filler comprises bismuth salt.

7. The device according to claim 1, wherein it comprises 80% polymer and 20% mineral filler.

8. The manufacturing procedure of a device according to claim 1, wherein it comprises a mixing phase of a medium-density polymer and mineral filler by compounding followed by an extrusion stage for products obtained by compounding.

9. The procedure according to claim 8, wherein the mixing step comprised by compounding comprises a preparation stage of elements to mix, this mixture comprising 81.5% lupolen 3721C, 15% BaSo4, 3% TiO2 and 0.5% colouring.

10. The procedure according to claim 8, wherein extrusion takes place according to four hot zones at 215° C., 226° C., 230° C. and 224° C. respectively with a screw rotating at 27.1 rotations/min.
